(19) United States
(12) Patent Application Publication
(10) Pub. No.: US 2023/0070220 A1
(43) Pub. Date: Mar. 9, 2023

(54) Balloon Expandable Delivery System with Uniform Inflation

(71) Applicant: BOSTON SCIENTIFIC SCIMED, INC., Maple Grove, MN (US)

(72) Inventors: James M. Anderson, Corcoran, MN (US); Joshua Stephan Havel, Champlin, MN (US); Adam David Grovender, Maple Grove, MN (US)

(73) Assignee: BOSTON SCIENTIFIC SCIMED, INC., MAPLE GROVE, MN (US)

(21) Appl. No.: 17/940,559

(22) Filed: Sep. 8, 2022

Related U.S. Application Data
(60) Provisional application No. 63/242,206, filed on Sep. 9, 2021.

(51) Int. Cl. A61F 2/24; A61M 25/10

(52) U.S. Cl. CPC A61F 2/2433; A61M 25/1025

(57) ABSTRACT

A delivery system is provided for delivering an expandable medical device. The delivery system includes a catheter shaft, a proximal stop coupled to the catheter shaft, an inner tube coupled to the proximal stop and extending distally therefrom, a distal stop coupled to the inner tube, and a balloon disposed over the distal stop, the inner tube, and the proximal stop. The inner tube defines an inflation lumen and has a plurality of openings extending through a sidewall of the inner tube into the inflation lumen. The distal stop includes at least one channel extending through a sidewall of the distal stop into the lumen. The distal stop is configured to allow inflation fluid to flow through the inner tube and the at least one channel into a distal region of the balloon.

### CROSS-REFERENCE TO RELATED APPLICATIONS

This application claims the benefit of priority of U.S. Provisional Application No. 63/242,206 filed Sep. 9, 2021, the entire disclosure of which is hereby incorporated by reference.

### TECHNICAL FIELD

The disclosure pertains to medical devices and more particularly to balloon expandable medical device delivery systems, and methods for using such medical devices.

### BACKGROUND

A wide variety of medical procedures and delivery systems have been developed for delivering and deploying balloon expandable medical devices. Some of these systems include stents and prosthetic heart valves with stent elements. These devices and systems may be used according to any one of a variety of methods. Of the known medical devices and methods, each has certain advantages and disadvantages. There is an ongoing need to provide alternative medical devices and systems as well as alternative methods for manufacturing and using medical devices.

### SUMMARY

This disclosure provides design, material, manufacturing method, and use alternatives for medical devices and systems. An example delivery system for an expandable medical device incudes a catheter shaft, a proximal stop coupled to the catheter shaft, an inner tube coupled to the proximal stop and extending distally therefrom, the inner tube defining an inflation lumen and having a plurality of openings extending through a sidewall of the inner tube into the inflation lumen, a distal stop coupled to the inner tube, the distal stop having a lumen and at least one channel extending through a sidewall of the distal stop into the lumen, and an inflatable balloon disposed over the distal stop, the inner tube, and the proximal stop, wherein the distal stop is configured to allow inflation fluid to flow through the inner tube and the at least one channel and into a distal end region of the balloon.

Alternatively, or additionally to the embodiment above, the at least one channel extends distally at an angle from the lumen in the distal stop to an opening in an outer surface of the distal stop.

Alternatively or additionally to any of the embodiments above, the opening is positioned under the distal end region of the balloon.

Alternatively or additionally to any of the embodiments above, a proximal end of the distal stop is flared outward defining a distal gap between an outer surface of the inner tube and an inner surface of the distal stop.

Alternatively or additionally to any of the embodiments above, the proximal end of the distal stop defines a plurality of spaced apart fingers angled radially outward from a longitudinal axis of the distal stop.

Alternatively or additionally to any of the embodiments above, the plurality of openings through the inner tube include proximal openings through a proximal end region of the inner tube, and distal openings through a distal end region of the inner tube.

Alternatively or additionally to any of the embodiments above, a central region of the inner tube between the proximal end region and the distal end region is devoid of any openings.

Alternatively or additionally to any of the embodiments above, a distal end of the proximal stop is flared outward defining a proximal gap between an outer surface of the inner tube and an inner surface of the proximal stop.

Alternatively or additionally to any of the embodiments above, a distal end of the inner tube is adjacent the at least one channel.

Alternatively or additionally to any of the embodiments above, at least some of the openings through the inner tube are positioned within the distal gap and some openings are positioned within the proximal gap.

Alternatively or additionally to any of the embodiments above, an outer surface of the inner tube defines a raised helical coil.

Alternatively or additionally to any of the embodiments above, an outer surface of the inner tube defines a plurality of longitudinal grooves.

Alternatively or additionally to any of the embodiments above, a proximal waist of the balloon is fixed to the catheter shaft and a distal waist of the balloon is fixed to the distal stop, distal of the at least one channel.

Alternatively or additionally to any of the embodiments above, the delivery system further includes a prosthetic heart valve crimped over the balloon onto the inner tube between the proximal and distal stops.

Another example delivery system for an expandable medical device includes a catheter shaft, a proximal stop coupled to the catheter shaft, an inner tube having a proximal end coupled to the proximal stop, the inner tube defining an inflation lumen, a distal stop coupled to a distal end of the inner tube, the distal stop having at least one channel extending through a sidewall of the distal stop, the at least one channel in fluid communication with the inflation lumen of the inner tube, and an inflatable balloon having a proximal end fixed to the catheter shaft and a distal end fixed to the distal stop, distal of the at least one channel, wherein the inner tube includes a plurality of openings through a sidewall of the inner tube, including proximal openings in a proximal end region of the inner tube at least partially covered by the proximal stop, and distal openings in a distal end region of the inner tube at least partially covered by the distal stop, wherein the proximal stop and the distal stop are configured to allow inflation fluid to flow from the inflation lumen, through the proximal openings, the distal openings, and through the at least one channel, to proximal and distal regions of the balloon for uniform inflation of the balloon.

Alternatively or additionally to any of the embodiments above, each channel extends distally at an angle from an inner surface of the distal stop to an opening in an outer surface of the distal stop.

Alternatively or additionally to any of the embodiments above, a proximal end of the distal stop is flared outward defining a distal gap between an outer surface of the inner tube and an inner surface of the distal stop, wherein the distal openings in the inner tube are positioned within the distal gap.

Alternatively or additionally to any of the embodiments above, a distal end of the proximal stop is flared outward defining a proximal gap between an outer surface of the inner tube and an inner surface of the proximal stop, wherein the proximal openings in the inner tube are positioned within the proximal gap.

Alternatively or additionally to any of the embodiments above, an outer surface of the inner tube defines a raised helical coil.

A further example delivery system for an expandable medical device includes a catheter shaft, a proximal stop coupled to the catheter shaft, an inner tube coupled to the proximal stop, the inner tube defining an inflation lumen and having a plurality of proximal openings extending through a sidewall of the inner tube, at least some of the proximal openings positioned under a distal end of the proximal stop in a proximal gap between an outer surface of the inner tube and an inner surface of the proximal stop, a distal stop having a lumen coupled to a distal end of the inner tube, the distal stop having at least one channel extending distally at an angle through a sidewall of the distal stop, and an inflatable balloon disposed over the distal stop, the inner tube, and the proximal stop, wherein the distal stop is configured to allow inflation fluid to flow through the inner tube and the at least one channel into a distal end region of the balloon.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example balloon expandable delivery system;

FIG. 2 is a perspective view of the distal stop of FIG. 1;

FIG. 3 is a cross-sectional view of the distal stop and nose cone of FIG. 1;

FIG. 4 is a cross-sectional view of the distal stop, nose cone, and part of the inner tube of FIG. 1;

FIG. 5 is a cross-sectional view of the system of FIG. 1 in a delivery configuration with a medical device clamped over the deflated balloon;

FIG. 6 is a cross-sectional view of the system of FIG. 5 in an expanded configuration;

FIGS. 7A, 7B, and 7C are perspective views of example inner tubes; and

FIG. 8 is a perspective view of another example inner tube.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Delivering balloon expandable medical devices such as stents or replacement heart valves requires inflating a balloon to achieve deployment of the medical device. Correct placement of the medical device may depend on uniform or symmetrical inflation of the balloon. Many balloon catheters rely on inflating the balloon from an inflation lumen ending at the proximal end of the balloon, which may result in the proximal end of the balloon inflating before the distal end. The medical device may thus be deployed in a proximal to distal orientation, which may result in an undesired placement of the medical device. Even with an inflation lumen having fluid ports disposed within the balloon may not achieve symmetrical deployment of a medical device clamped or crimped onto the inflation lumen, as the device may block inflation ports underneath the device, resulting in the proximal end of the balloon inflating first. In particular, when deploying a replacement heart valve, such as in transcatheter aortic valve implantation (TAVI), proper alignment is desired. Asymmetrical balloon inflation may result in misalignment of the valve during deployment.

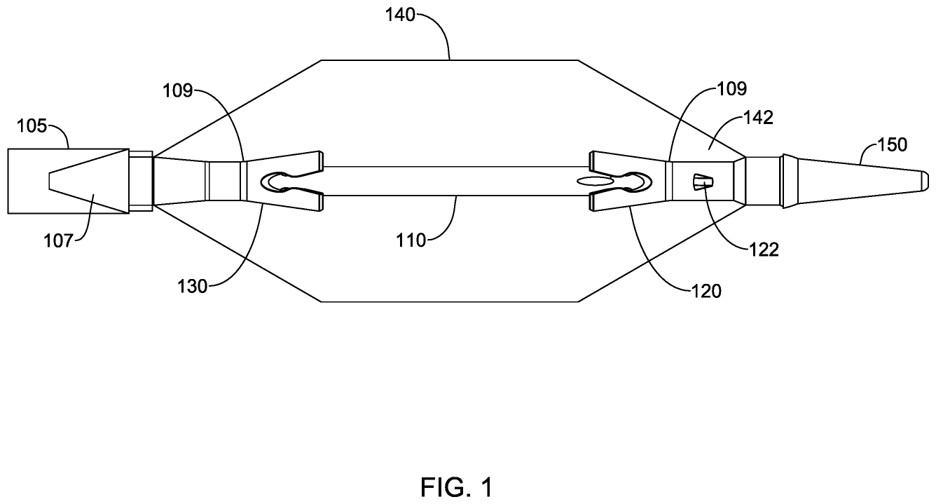

FIG. 1 illustrates some elements of an example delivery system 100 for deploying a balloon expandable medical device. The delivery system 100 may include a catheter shaft 105, a proximal stop 130 coupled to the catheter shaft, an inner tube 110 coupled to the proximal stop 130, and a distal stop 120 coupled to the distal end of the inner tube 110. A nose cone 150 may be coupled to the distal end of the distal stop 120. An inflatable balloon 140 may be disposed over the distal stop 120, the inner tube 110, and the proximal stop 130. The distal stop 120 may include at least one channel 122 configured to deliver inflation fluid to a distal end region 142 of the balloon 140. A distal neck of the balloon 140 may be coupled to the distal stop 120 at a location distal of the channel 122. In some embodiments, the distal neck of the balloon 140 may be coupled to an enlarged distal end region 127 of the distal stop 120. A proximal strain relief element 107 may be coupled to the proximal end of the proximal stop 130 and the catheter shaft 105. A proximal neck of the balloon 140 may be coupled to the strain relief element 107 or the catheter shaft 105. In some embodiments, one or more marker band 109 may be provided on the distal stop 120, the proximal stop 130 or any other region of the delivery system.

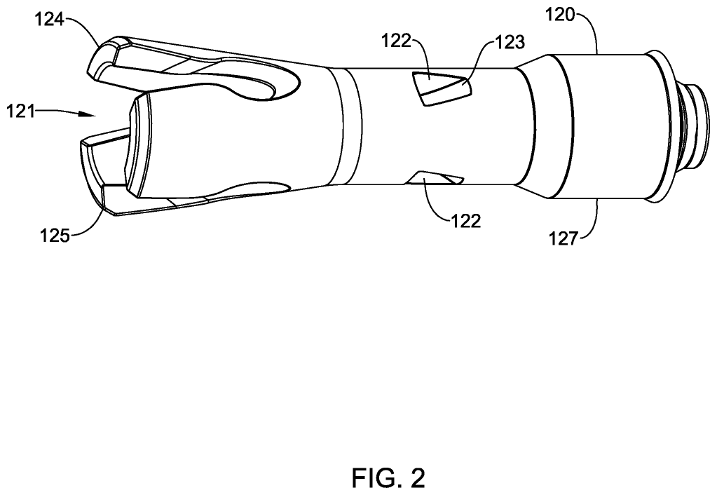

Details of the distal stop 120 are illustrated in FIG. 2. The distal stop 120 may define a lumen 121 configured to receive a distal end of the inner tube 110, and at least one channel 122 extending from an opening 123 in the outer surface, through the sidewall and into the lumen 121. In the example shown in FIG. 2, the distal stop 120 has three channels 122 spaced apart around the circumference. The distal stop 120 may have an enlarged distal end region 127 to which the distal neck of the balloon may be bonded. The proximal end 124 of the distal stop 120 may be flared outward, and may include a plurality of spaced apart fingers 125 angled radially outward from a longitudinal axis of the distal stop. In some embodiments, the fingers 125 may be compressible, and may be made of a soft, elastomeric material. In one example, the distal stop 120 including the fingers 125, may be made of a material having a Shore D of 65-75. An example material is flexible polyurethane (FPU 50), often used in 3D printing, with a Shore D of 71.

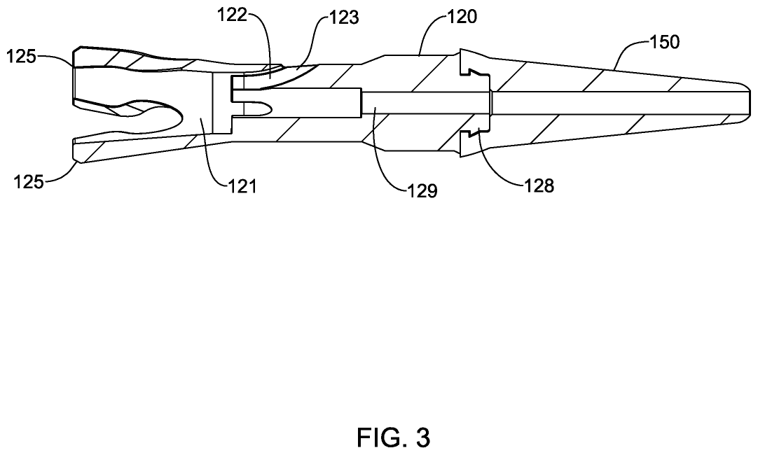

The cross-sectional view in FIG. 3 illustrates the lumen 121 and one channel 122 of the distal stop 120 shown in FIG. 2. Each of the channels 122 may extend distally at an angle from the lumen 121 to the opening 123 in the outer surface of the distal stop 120. The distal stop 120 may also define a guide wire lumen 129 extending along the length of the distal stop. The distal end may include an engagement element 128 configured to engage the nose cone 150. In some embodiments, the engagement element 128 includes one or more ridge configured to provide a snap fit with the nose cone 150. In other embodiments, the engagement element 128 may include threading configured to mate with threading on the nose cone. In addition to or alternatively to any snap fit or threaded connection, adhesive or a polymer bond may be used to fix the nose cone 150 onto the distal stop 120.

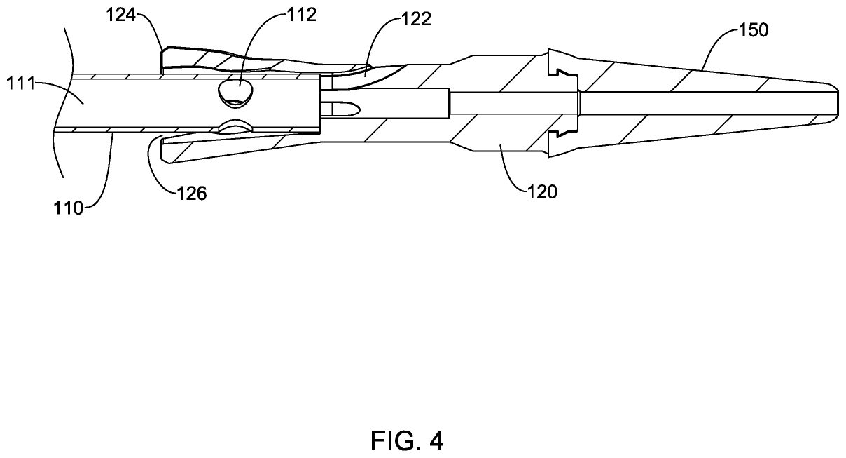

In FIG. 4, the distal end of the inner tube 110 is shown inserted into the lumen of the distal stop 120 with the distal end of the inner tube adjacent the channel 122. The inner tube 110 may define an inflation lumen 111 and a plurality of openings 112 extending through the sidewall and into the inflation lumen 111. The channels 122 may be in fluid communication with the distal end of the inflation lumen 111, allowing inflation fluid to exit the distal end of the inner tube 110 and move through the channels 122 and into the distal end region 142 of the balloon. The flared proximal end 124 of the distal stop may define a distal gap 126 between an outer surface of the inner tube 110 and an inner surface of the distal stop. In some embodiments, at least some of the openings 112 may be at least partially covered by the distal stop, however the distal gap 126 may provide space for inflation fluid exiting the openings 112 to flow proximally along the outer surface of the inner tube 110 and into the balloon.

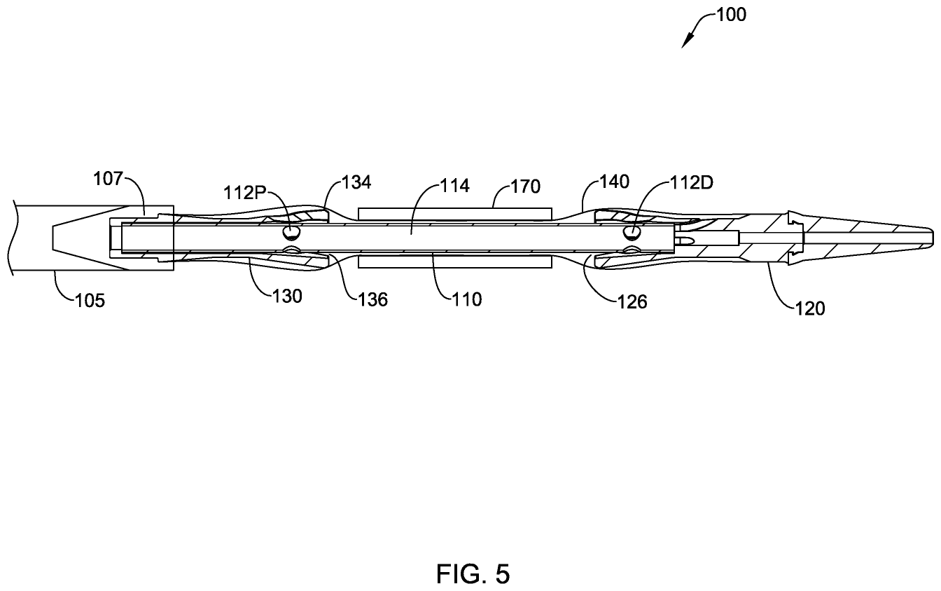

FIG. 5 illustrates the delivery system 100 with the balloon 140 deflated and a medical device 170 compressed over the balloon and onto the inner tube 110 between the proximal stop 130 and the distal stop 120. The distal stop 120 and the proximal stop 130 are not configured to overlap and/or retain the medical device 170. The medical device 170 is held in place by being crimped or compressed around the balloon 140 and inner tube 110. The proximal end of the inner tube 110 may be disposed within the proximal stop 130. The plurality of openings 112 through the inner tube 110 may include proximal openings 112P through a proximal end region of the inner tube, and distal openings 112D through a distal end region of the inner tube. A distal end 134 of the proximal stop 130 may be flared outward defining a proximal gap 136 between an outer surface of the inner tube 110 and an inner surface of the proximal stop 130. In some embodiments, at least some of the proximal openings 112P may be at least partially covered by the proximal stop 130, however the proximal gap 136 may provide space for inflation fluid exiting the proximal openings 112P to flow distally along the outer surface of the inner tube 110 and into the balloon. At least some of the distal openings 112D through the inner tube may be positioned within the distal gap 126 and some proximal openings 112P may be positioned within the proximal gap 136. In some embodiments, a central region 114 of the inner tube 110 between the proximal and distal end regions of the inner tube may be devoid of any openings.

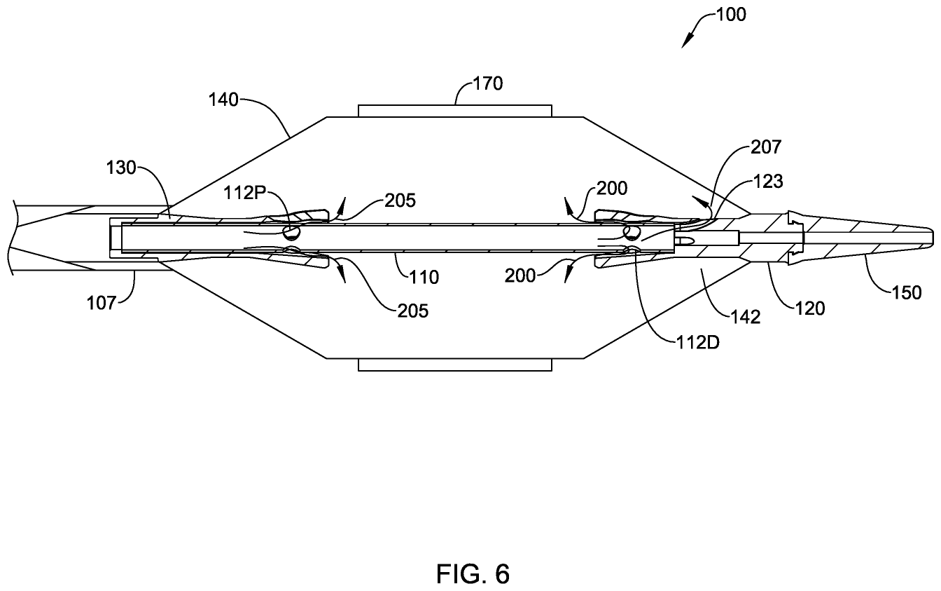

The direction of inflation fluid moving through the inner tube 110 and into the balloon 140 during inflation is illustrated in FIG. 6. Arrows 200 show the direction of inflation fluid exiting the distal openings 112D and moving through the distal gap and into the balloon. Arrows 205 show the direction of inflation fluid exiting the proximal openings 112P and moving through the proximal gap and into the balloon. Arrow 207 shows the direction of inflation fluid flowing out of the channel opening 123, which may be positioned under the distal end region 142 of the balloon. The combination of distal openings 112D and channel 122 provide inflation fluid to the distal end of the balloon. This distally directed inflation fluid, in addition to the inflation fluid exiting the proximal openings 112P, may cause the balloon 140 to be inflated symmetrically, with the distal and proximal regions of the balloon being inflated simultaneously.

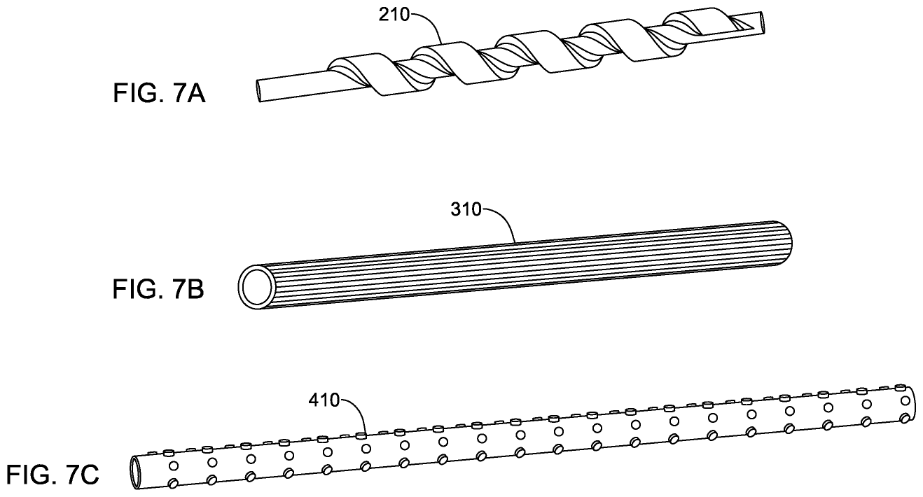

The outer surface of the inner tube 110 as illustrated in FIGS. 1 and 4-6 is smooth, however the outer surface may have any surface structure. FIGS. 7A-7C illustrate various surface structures for the inner tube 110. In some embodiments, the outer surface of the inner tube 210 may define a raised helical coil, as shown in FIG. 7A. The raised coil may provide improved device retention of the compressed medical device. The raised coil may also provide increased column strength and compression resistance, such that when a medical device 170 is compressed over the inner tube 210, the inner tube is not crushed. In the embodiment shown in FIG. 7B, the outer surface of the inner tube 310 defines a plurality of longitudinal grooves. In addition to providing increased column strength and compression resistance, the grooves may act as channels for directing flow of inflation fluid. FIG. 7C illustrates an embodiment in which the outer surface 410 of the inner tube defines a plurality of bumps or protrusions. The plurality of grooves and the plurality of bumps or protrusions may provide increased column strength and compression resistance, and prevent the inner tube being damaged during compression of the medical device onto the system.

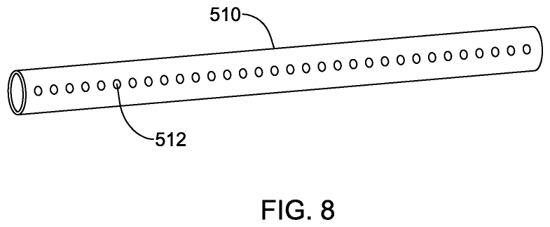

In addition to or alternatively to the surface structures shown in FIGS. 7A-7C, the inner tube 510 may have a plurality of openings 512 extending through the sidewall and into the lumen. These openings 512 may be in addition to or alternatives to the openings 112 in the inner tube 110 discussed above. The openings 512 may extend along the entire length of the inner tube 510, as illustrated in FIG. 8, or they may be present only on the distal and/or end regions.

It will be understood that any dimensions and angles described in association with the above examples are illustrative only, and that other dimensions and angles of the transition zone are contemplated. The materials that can be used for the various components of the delivery system (and/or other systems or components disclosed herein) and the various elements thereof disclosed herein may include those commonly associated with medical devices. For simplicity purposes, the following discussion makes reference to the delivery system 100 (and variations, systems or components disclosed herein). However, this is not intended to limit the devices and methods described herein, as the discussion may be applied to the other elements, members, components, or devices disclosed herein.

In some embodiments, the distal stop 120, inner tube 110, proximal stop 130, and proximal strain relief element 107 may be made of a material having a Shore D of 71, such as flexible polyurethane (FPU 50), often used in 3D printing. The distal nose cone 150, and in some embodiments the inner tube 110, may be made of a thermoplastic polyurethane having a Shore A of 70, such as polyurethane elastomer EPU 40.

In some embodiments, portions of the delivery system 100 (and variations, systems or components thereof disclosed herein) may be made from a metal, metal alloy, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the delivery system 100 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the delivery system 100 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery system 100 (and variations, systems or components thereof disclosed herein) to achieve the same result.

In some embodiments, the delivery system 100 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A delivery system for an expandable medical device, comprising:
a catheter shaft;
a proximal stop coupled to the catheter shaft;
an inner tube coupled to the proximal stop and extending distally therefrom, the inner tube defining an inflation lumen and having a plurality of openings extending through a sidewall of the inner tube into the inflation lumen;
a distal stop coupled to the inner tube, the distal stop having a lumen and at least one channel extending through a sidewall of the distal stop into the lumen; and
an inflatable balloon disposed over the distal stop, the inner tube, and the proximal stop;
wherein the distal stop is configured to allow inflation fluid to flow through the inner tube and the at least one channel into a distal end region of the balloon.

2. The delivery system of claim 1, wherein each channel extends distally at an angle from the lumen in the distal stop to an opening in an outer surface of the distal stop.

3. The delivery system of claim 2, wherein the opening is positioned under the distal end region of the balloon.

4. The delivery system of claim 1, wherein a proximal end of the distal stop is flared outward defining a distal gap between an outer surface of the inner tube and an inner surface of the distal stop.

5. The delivery system of claim 4, wherein the proximal end of the distal stop defines a plurality of spaced apart fingers angled radially outward from a longitudinal axis of the distal stop.

6. The delivery system of claim 5, wherein the plurality of openings through the inner tube include proximal openings through a proximal end region of the inner tube, and distal openings through a distal end region of the inner tube.

7. The delivery system of claim 6, wherein a central region of the inner tube between the proximal end region and the distal end region is devoid of any openings.

8. The delivery system of claim 6, wherein a distal end of the proximal stop is flared outward defining a proximal gap between an outer surface of the inner tube and an inner surface of the proximal stop.

9. The delivery system of claim 8, wherein a distal end of the inner tube is adjacent the at least one channel.

10. The delivery system of claim 8, wherein at least some of the openings through the inner tube are positioned within the distal gap and some openings are positioned within the proximal gap.

11. The delivery system of claim 1, wherein an outer surface of the inner tube defines a raised helical coil.

12. The delivery system of claim 1, wherein an outer surface of the inner tube defines a plurality of longitudinal grooves.

13. The delivery system of claim 1, wherein a proximal waist of the balloon is fixed to the catheter shaft and a distal waist of the balloon is fixed to the distal stop, distal of the at least one channel.

14. The delivery system of claim 1, further comprising a prosthetic heart valve crimped over the balloon onto the inner tube between the proximal and distal stops.

15. A delivery system for an expandable medical device, comprising:
a catheter shaft;
a proximal stop coupled to the catheter shaft;
an inner tube having a proximal end coupled to the proximal stop, the inner tube defining an inflation lumen;
a distal stop coupled to a distal end of the inner tube, the distal stop having at least one channel extending through a sidewall of the distal stop, the at least one channel in fluid communication with the inflation lumen of the inner tube; and
an inflatable balloon having a proximal end fixed to the catheter shaft and a distal end fixed to the distal stop, distal of the at least one channel;
wherein the inner tube includes a plurality of openings through a sidewall of the inner tube, including proximal openings in a proximal end region of the inner tube at least partially covered by the proximal stop, and distal openings in a distal end region of the inner tube at least partially covered by the distal stop;
wherein the proximal stop and the distal stop are configured to allow inflation fluid to flow from the inflation lumen, through the proximal openings, the distal openings, and through the at least one channel, into proximal and distal regions of the balloon for uniform inflation of the balloon.

16. The delivery system of claim 15, wherein each channel extends distally at an angle from an inner surface of the distal stop to an opening in an outer surface of the distal stop.

17. The delivery system of claim 15, wherein a proximal end of the distal stop is flared outward defining a distal gap between an outer surface of the inner tube and an inner surface of the distal stop, wherein the distal openings in the inner tube are positioned within the distal gap.

18. The delivery system of claim 17, wherein a distal end of the proximal stop is flared outward defining a proximal gap between an outer surface of the inner tube and an inner surface of the proximal stop, wherein the proximal openings in the inner tube are positioned within the proximal gap.

19. The delivery system of claim 15, wherein an outer surface of the inner tube defines a raised helical coil.

20. A delivery system for an expandable medical device, comprising:
a catheter shaft;
a proximal stop coupled to the catheter shaft;
an inner tube coupled to the proximal stop, the inner tube defining an inflation lumen and having a plurality of proximal openings extending through a sidewall of the inner tube, at least some of the proximal openings positioned under a distal end of the proximal stop in a proximal gap between an outer surface of the inner tube and an inner surface of the proximal stop;
a distal stop having a lumen coupled to a distal end of the inner tube, the distal stop having at least one channel extending distally at an angle through a sidewall of the distal stop; and
an inflatable balloon disposed over the distal stop, the inner tube, and the proximal stop;
wherein the distal stop is configured to allow inflation fluid to flow through the inner tube and the at least one channel into a distal end region of the balloon.
